(19) United States
(12) Patent Application Publication
(10) Pub. No.: US 2009/0062829 A1
(43) Pub. Date: Mar. 5, 2009

(54) Microsurgical Cutting Instrument for Refractive Ophthalmological Treatments

(75) Inventors: Christof Donitzky, Eckental (DE); Daniel Thimm, Schwanstetten (DE)

Correspondence Address: HAYNES AND BOONE, LLP;IP Section, 2323 Victory Avenue, Suite 700, Dallas, TX, 75219, US

(73) Assignee: Wavelight AG, Erlangen (DE)

(21) Appl. No.: 12/064,486

(22) PCT Filed: Aug. 10, 2006
(86) PCT No.: PCT/EP2006/007929

§ 371 Date: Nov. 3, 2008

(30) Foreign Application Priority Data
Aug. 24, 2005 (EP) 05018391.2

(51) Int. Cl. A61F 9/007

(52) U.S. Cl. 606/166

(57) ABSTRACT

A surgical cutting instrument for refractive opthalmological treatments includes an instrument base body unit (12) to be placed on the eye and that can be fixed relative to the latter, wherein guide holding means (30, 44) are associated with the instrument base body unit, the said means being intended and designed for the movably guided holding, relative to the instrument base body unit, of at least two cutting units (14) of different cutting geometries.

### CROSS REFERENCE

This application was originally filed as Patent Cooperation Treaty Application Number PCT/EP2006/007929 filed Aug. 10, 2006, which claims priority of European Application Number 05018391.2, filed Aug. 24, 2005.

### CROSS-REFERENCE TO RELATED APPLICATIONS

This application is a United States national phase application of co-pending international patent application number PCT/EP2006/007929, filed Aug. 10, 2006, the disclosure of which is incorporated herein by reference.

### BACKGROUND

The present invention relates in general to the field of refractive treatment of the eye, in particular the human eye, in order to treat defective vision.

In order to treat low-order types of defective vision such as myopia, hyperopia, astigmatism, myopic astigmatism and hyperopic astigmatism, inter alia the so-called LASIK (LASer In situ Keratomileusis) and so-called LASEK (LASer Epithelial Keratomileusis) methods have been established. In both methods a flap with a diameter of for example about 8 to 10 mm is stripped from the surface of the cornea as far as a small remaining part serving as hinge. This flap is folded to one side, the corneal material lying thereunder thereby being made accessible for refractive laser treatment. After the treatment the flap is folded back again. Within the scope of the laser treatment, which is normally carried out with an excimer laser at a wavelength of for example 193 nm, material is ablated (removed) from the corneal stroma according to an ablation profile that was determined beforehand and possibly adapted during the treatment. The reshaping of the cornea achieved in this way alters the refractive properties of the cornea and thus of the optical system of the eye as a whole. On account of the fact that the flap is folded back, no externally open wound remains and the healing process is as a rule relatively quick.

In the LASIK method the flap is produced by means of a so-called microkeratome, which shaves off the flap from the surface of the cornea. The microkeratome has a cutting head, which is normally moved linearly over the cornea. The cutting head is loaded with a planar cutting blade, which is adjusted to a specific setting angle (angle of attack) relative to an applanation surface of the cutting head and projects by an amount, determined by the desired flap thickness, over the applanation surface. If the cutting head is moved over the cornea, the cutting blade cuts into the cornea and thereby forms the flap. In addition to the feed movement of the cutting head the cutting blade is normally caused to execute lateral oscillations.

In the LASIK method it was for a long time normal practice to produce the flap with a thickness of about 100 μm to 200 μm. In this case the cut goes through the approximately 40 μm to 60 μm thick corneal epithelium and the approximately 8 μm to 15 μm thick Bowman membrane lying underneath, directly into the stroma, the thickness of which is for example about 400 μm to 500 μm.

In DE 103 17 972 B3 it is proposed to adjust the cutting angle and the cutting radius of the cutting blade so that the blade is on the one hand sharp enough to fully penetrate the corneal epithelium, but on the other hand is not so sharp as to penetrate also the Bowman membrane. With such a blade the epithelium should be able to be cleanly separated from the Bowman membrane, without any epithelial cells remaining and without the Bowman membrane being damaged. The flap that is formed is thus a pure epithelial flap, which is only about 50 μm thick.

In the LASIK method the problem arises that the cornea can, on account of its elasticity, evade the cutting blade when the latter is inserted from the side. This can result in an irregular flap edge and a not uniquely reproducible flap size. With a relatively blunt cutting edge, as is proposed in DE 103 17 972 B3, this process can be particularly serious.

In the LASEK method similarly only the epithelium is separated from the cornea. The flap size is defined by means of a so-called microtrepan, which is a cutting element with a circular cutting edge running along a circular arc, wherein the cutting edge does not extend over a full circle but only over part of the circumference of a circle, for example about 250° to 300°. A gap is provided in the remaining circumferential region. The trepan is placed on the eye and, when rotated clockwise and anticlockwise by a limited small angle of rotation of for example about 10°, its circular cutting edge penetrates into the cornea. A circular cut is made in the epithelium, the length of which is equal to that of the circular cutting edge plus the angle of rotation of the trepan. The region that has not been cut forms the hinge of the flap. For the cut the trepan is guided in a guide body with a cylindrical receptacle opening, into which the trepan is inserted.

After the cutting with the trepan the epithelial tissue is softened by means of an alcoholic solution that is dripped into a cylinder placed on the eye, until the flap can be lifted with a ductor or spatula from the Bowman membrane and moved to one side. The use of the alcohol solution has disadvantages however, since it can kill the cells of the basal membrane lying between the epithelium and Bowman membrane. This delays the closure of the wound, since new epithelial cells can grow only slowly.

Cutting instruments for the LASIK and LASEK methods generally include an instrument base body unit that is placed on the eye and can be fixed relative to the eye. In order to fix the instrument base body unit on the eye it is known to provide the unit with a suction ring, which is placed on the limbus and held on the latter under suction produced by a vacuum. For this purpose the instrument base body unit includes an evacuation connection, which can be connected via a hose line system to an external vacuum pump and is joined, via an evacuation path system formed in the instrument base body unit, to an annular evacuation groove provided on the lower side of the suction ring facing towards the eye.

Previous cutting instruments were designed either specifically for the LASIK method or specifically for the LASEK method.

### SUMMARY

The object of the present invention is to provide a microsurgical cutting instrument with which improved treatment results can be achieved compared to the previously employed LASEK and LASIK methods.

To achieve this object a microsurgical cutting instrument for refractive opthalmological treatments is proposed according to the invention, with an instrument base body unit to be placed on the eye and which can be fixed relative to the latter, wherein guide holding means are associated with the instrument base body unit, which means are intended and designed for the movably guided holding, relative to the instrument base body unit, of at least two cutting units with different cutting geometries. In particular the guide holding means can include at least a first guide formation for guiding a first cutting unit and at least a second guide formation, different from the first guide formation, for guiding a second cutting unit.

With a cutting instrument according to the invention the advantages of the conventional LASEK and LASIK techniques can be combined in a single instrument. Thus, the instrument base body unit is loaded or can be loaded with a first cutting unit, which enables a cut to be made which defines only the flap edge, without penetrating underneath the epithelium and lifting the latter. The instrument base body unit is furthermore loaded or can be loaded with a second cutting unit, which enables the instrument to engage with the cut made by the first cutting unit and remove the flap from the corneal material lying underneath. The use of an alcohol solution can thus be dispensed with, and at the same time it is possible to make flaps of reproducible size with a clean, uniform edge. Conveniently the first cutting unit has a circular cutting edge, wherein the at least one first guide formation is designed for the rotatory guidance of the first cutting unit, while the second cutting unit has a rectilinear cutting edge and the at least one second guide formation is designed for the at least approximately linear guidance of the second cutting unit.

Since the flap edge is uniquely defined by the first cut, the cutting edge of the second cutting unit can be comparatively blunt. In particular it can be sufficiently blunt that essentially there is no danger that it will cut into the Bowman membrane and thereby reach the corneal stroma. The cutting edge of the second cutting unit must simply be sufficiently sharp that it can remove the epithelium from the Bowman membrane. In contrast to the method discussed in DE 103 17 972 B3, the cutting edge of the second cutting unit does not itself have to cut into the epithelium from outside. It can instead utilise the circular cut made beforehand by the first cutting unit as an “entry”, and is then as it were guided in the circular cut.

In a preferred embodiment the guide holding means are designed so that they allow the simultaneous arrangement of both cutting units on the instrument base body unit.

The guide holding means can include a guide body separate from the instrument base body unit but positionable on the latter, on which guide body is formed the at least one first guide formation, while the at least one second guide formation is formed on the instrument base body unit. For the cutting operation of the second cutting unit the guide body can in this case be removed from the instrument base body unit.

The guide body preferably comprises an approximately cylindrical guide receptacle opening, in which the first cutting unit can be accommodated and can rotate about the cylindrical axis. For this purpose the guide body and the instrument base body unit advantageously comprise co-operating rotation prevention means, which prevent the guide body rotating relative to the instrument base body unit about the cylindrical axis.

The cutting edge of the second cutting unit can be formed by a planar cutting blade, a setting angle of the cutting blade of between 18° and 32°, preferably between 21° and 28° and most particularly preferably of about 25° with respect to the guidance direction of the second cutting unit being preferred.

The invention furthermore relates to a method for forming a corneal flap on the eye, this method including the following steps:
making a circular cut in the cornea by means of a first cutting unit comprising a circular cutting edge,
inserting a rectilinear cutting edge of a second cutting unit into the cornea, starting from the circular cut, so as to form the flap.

In particular this method can be implemented with a cutting instrument of the type described hereinbefore, in which the first and the second cutting unit can be used in succession without temporarily removing the instrument base body unit from the eye.

The circular cut preferably extends only so deep in the cornea that a ca. 50 μm thick epithelial flap can be removed from the surface of the cornea without damaging the Bowman membrane. As previously mentioned, in this connection the sharpness of the rectilinear cutting edge is preferably such that there is essentially no danger of damaging the Bowman membrane when the rectilinear cutting edge cuts into the cornea.

The scope of the invention also covers a purposefully blunt cutting element with a rectilinear cutting edge of circular cross-section, which is intended for use in a cutting instrument of the type described beforehand and/or for use within the scope of the method described beforehand. According to the invention the cutting edge of such a cutting element has a radius of curvature of between 1 and 10 μm, preferably between 3 and 8 μm and most particularly preferably between 4 and 6 μm. Also, the cutting element in a first cutting region immediately adjoining the cutting edge has a cutting angle of between 15° and 22°, preferably between 16° and 20° and most preferably of about 18°.

In a second cutting region adjoining the first cutting region the cutting element can have a cutting angle between 10° and 18°, preferably between 11° and 16° and most preferably of about 13°, in which the cutting angle of the second cutting region is smaller than the cutting angle of the first cutting region. If desired the cutting element can furthermore have, in a third cutting region adjoining the second cutting region, a cutting angle between 7° and 15°, preferably between 8° and 12° and most preferably of about 9°, wherein the cutting angle of the third cutting region is smaller than the cutting angle of the second cutting region.

### BRIEF DESCRIPTION OF THE DRAWINGS

The invention is described in more detail hereinafter with the aid of the accompanying drawings, in which:

FIG. 1 is a perspective view of a microsurgical cutting instrument—loaded with a trepan—according to an example of implementation of the invention,

FIG. 2 is a section through the cutting instrument of FIG. 1 in a state in which it is placed on an eye,

FIG. 3 is a perspective view of a cutting head used with the cutting instrument of FIG. 1,

FIG. 4 is an enlarged sectional representation of the region of the cutting head of FIG. 3, in which the cutting blade unit is arranged,

FIG. 5 is a perspective view of the cutting blade unit used with the cutting head of FIG. 3,

FIGS. 6 and 7 show in highly schematic form two successive phases of the method according to the invention for the refractive treatment of an eye,

FIG. 8 is a perspective view of the cutting instrument of FIGS. 1 and 2—with slight modifications—in an operationally ready state, in which it is loaded with a trepan and a cutting head, and

FIG. 9 shows on an enlarged scale a part of a cutting blade close to the cutting edge, that can be used with the cutting head of FIGS. 4 and 5.

### DETAILED DESCRIPTION

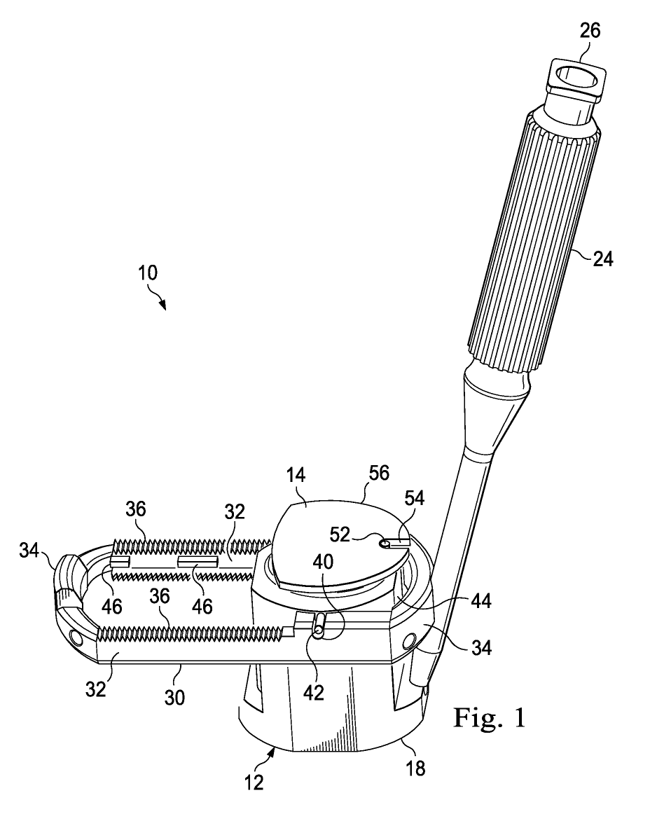

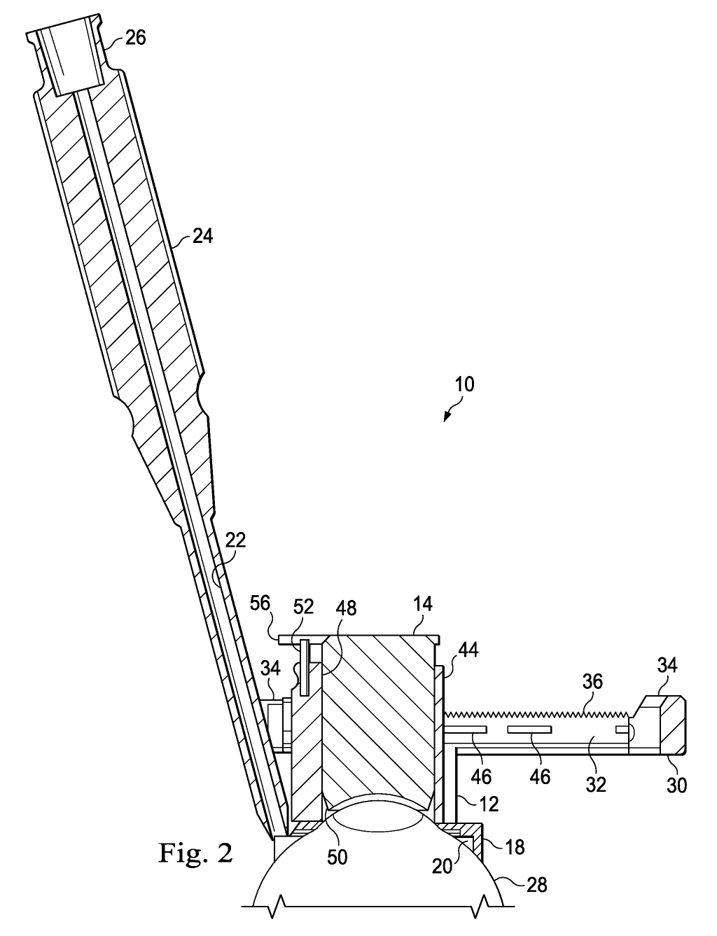

Reference will first of all be made to FIGS. 1 and 2. The microsurgical cutting instrument illustrated there is generally identified by the reference numeral 10. It is intended for microsurgical treatments on the eye, in particular the human eye, aimed at reshaping the cornea in order to correct defective vision. Within the scope of such a treatment an epithelial flap is first of all removed from the surface of the cornea by means of the cutting instrument 10. After the epithelial flap has been folded to one side, a treatment with a refractively operating laser is then carried out, in which material is ablated from the corneal stroma. After completion of the laser treatment the epithelial flap is folded back.

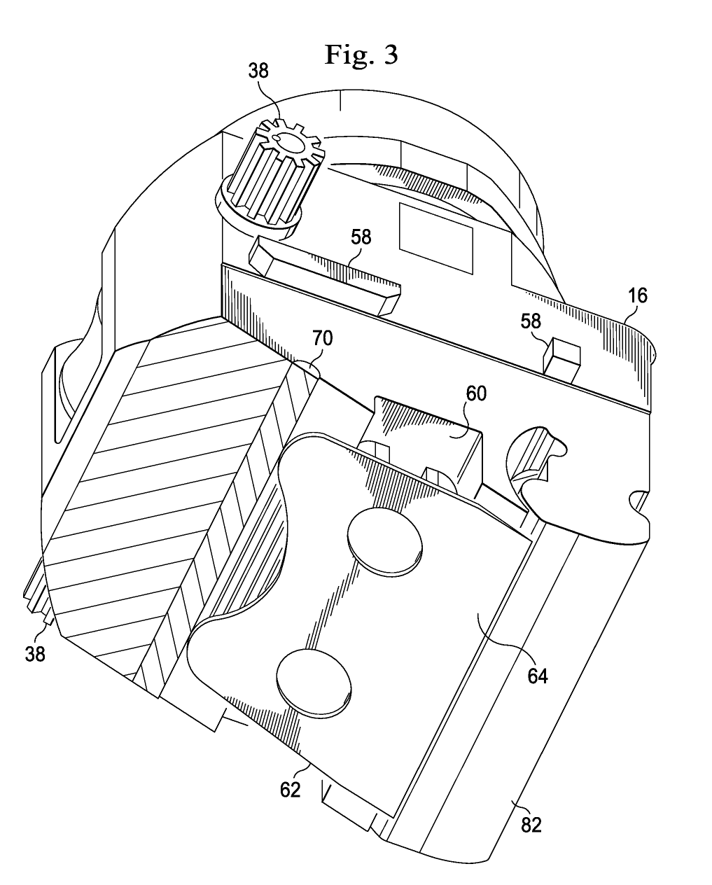

The cutting instrument 10 comprises an instrument base body 12 made for example of stainless steel, on which a trepan 14 forming a first cutting unit and a cutting head 16 (FIG. 3) forming a second cutting unit can alternately be mounted. The instrument base body forms a suction ring 18 with an evacuation groove 20, which is part of an evacuation path system 22 that extends through an elongated gripping piece 24 of the cutting instrument 10 as far as an evacuation connection 26. A flexible hose line can be attached to the evacuation connection 26 in a manner not described in more detail, which hose line can be connected to a vacuum pump installed in a base station of a device for refractive opthalmological treatment. To fix the instrument base body 12 to the eye the suction ring 18 is placed on the limbus (edge of the cornea), and by applying a vacuum to the evacuation path system 22 a suction effect is produced which sucks the suction ring 18 onto the eye identified by the reference numeral 28 in FIG. 2.

The instrument base body 12 furthermore comprises a guide and holding frame 30, connected in particular in one piece to the suction ring 18, which consists of two rectilinear longitudinal struts 32 extending spaced apart and parallel to one another, and two arcuately curved connection pieces 34 joining the longitudinal struts 32 to one another at their ends. On their upper side facing away from the eye the longitudinal struts 32 are formed having a toothed section 36 on a part of their longitudinal extension, which serves for toothed engagement with drive pinions 38 (FIG. 3) of the cutting head 16. In the tooth-free region of their upper side facing away from the eye the longitudinal struts 32 furthermore comprise in each case a notch 40, which serves to accommodate a positioning pin 42 of a guide sleeve 44 for the trepan 14. In addition rib pieces 46 are formed on the insides of the longitudinal struts 32 facing towards one another, which serve for the linearly movable guidance of the cutting head 16.

The guide sleeve 44 is a structural part separate from the instrument base body 12 and serves for the rotationally movable guidance of the trepan 14. The sleeve is inserted between the two longitudinal struts 32 in a region above the suction ring 18 and is supported on the one hand downwardly (referred to the direction of view in FIGS. 1 and 2) by the engagement of its positioning pin 42 in the notches 40, and on the other hand is prevented from rotating relative to the instrument base body 12. The sleeve has a circular cylindrical internal circumferential surface 48, which serves as a guide surface for the rotationally movable guidance of the trepan 14. The trepan 14 has, as illustrated in FIG. 2, on its lower side, i.e. the side facing in the position of use towards the eye 28, a cutting edge 50 extending in the form of a circular arc, which extends in the circumferential direction over for example an angle of about 250° to 300°. On the remainder of the circular circumference the cutting edge 50 of the trepan 14 is recessed in a manner known per se, so that there is no cutting contact with the cornea of the eye 28 in the recessed region. On account of its extension along a circular arc the cutting edge 50 is in this case also termed a circular cutting edge. The roundness of the cutting edge 50 constitutes a first cutting edge geometry in the context of the invention. It should be noted in this connection that the expression “round” in connection with the cutting edge 50 does not refer to the cutting edge cross-section (i.e. the cutting edge radius), but simply—as already mentioned above—to the circular arcuate curved contour of the cutting edge 50.

In the illustration shown in FIG. 2 the trepan 14 is shown as a solid body. It is understood however that alternatively a tubular or sleeve-shaped body can be used as trepan.

The trepan 14 is rotatable about a restricted angle of rotation relative to the guide sleeve 44. An axially projecting pin 52 provided on the guide sleeve 44 in the example shown in FIGS. 1 and 2, which engages in a recess 54 formed in a radially (referred to the axis of the guide sleeve 44) distant collar 56 of the trepan 14 serves to restrict the angle of rotation. The circumferential width of the recess 54 determines the relative angle of rotation by which the trepan 14 can rotate with respect to the guide sleeve 44. It is understood that in a modified form of implementation the guide sleeve 44 can have a recess restricting the angle of rotation, in which a pin of the trepan 14 can engage. Other forms of stop means that are effective between the guide sleeve 44 and the trepan 14 and which limit the angle of rotation are also possible.

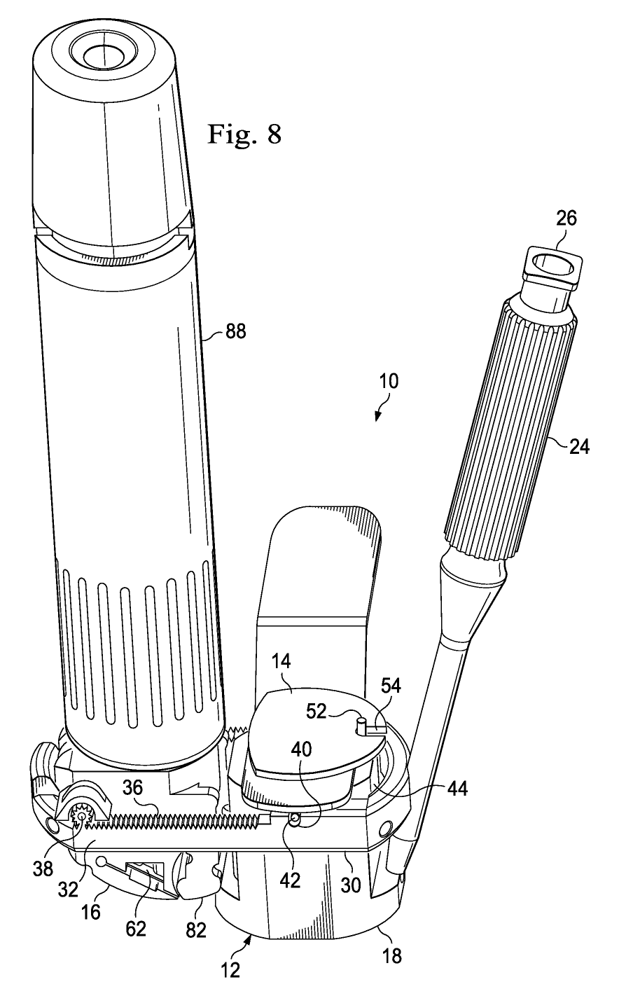

The cutting head 16 and the guide sleeve 44 with the trepan 14 inserted therein can be arranged simultaneously on the instrument base body 12. This state of the cutting instrument is shown in FIG. 8. The instrument base body 12 can therefore be loaded with both cutting units, i.e. the trepan 14 and cutting head 16, already before the operation. To use the trepan 14 the cutting head 16 can remain on the instrument base body 16; the cutting head 16 does not interfere in this connection. However, to use the cutting head 16 the guide sleeve 44 must be removed beforehand from the instrument base body 12, since otherwise the cutting head 16 could not be moved forwardly over the eye.

The cutting head 16 is mounted on the instrument base body 12 by inserting it in the rear region of the guide and holding frame 30 between the longitudinal struts 32. As shown in FIG. 3, the cutting head 16 also has rib pieces 58 on its two lateral sides, which rib pieces then engage with the rib pieces 46 of the frame 30 and move along the latter when the cutting head 16 is correctly attached to the instrument base body 12. In this correctly inserted state the drive pinions 38 of the cutting head 16 engage in a comb-like manner in the teeth 36 of the longitudinal struts 32. The cutting head 16 can be coupled in a manner not shown in more detail to electric motor drive means, by means of which the drive pinions 38 can be driven. When the drive pinions 38 are driven the cutting head 16 consequently moves along the longitudinal struts 32, whereby a cutting blade unit 62 accommodated in a receiving pocket 60 of the cutting head 16 moves over the cornea of the eye to be treated and thus removes a flap from the cornea.

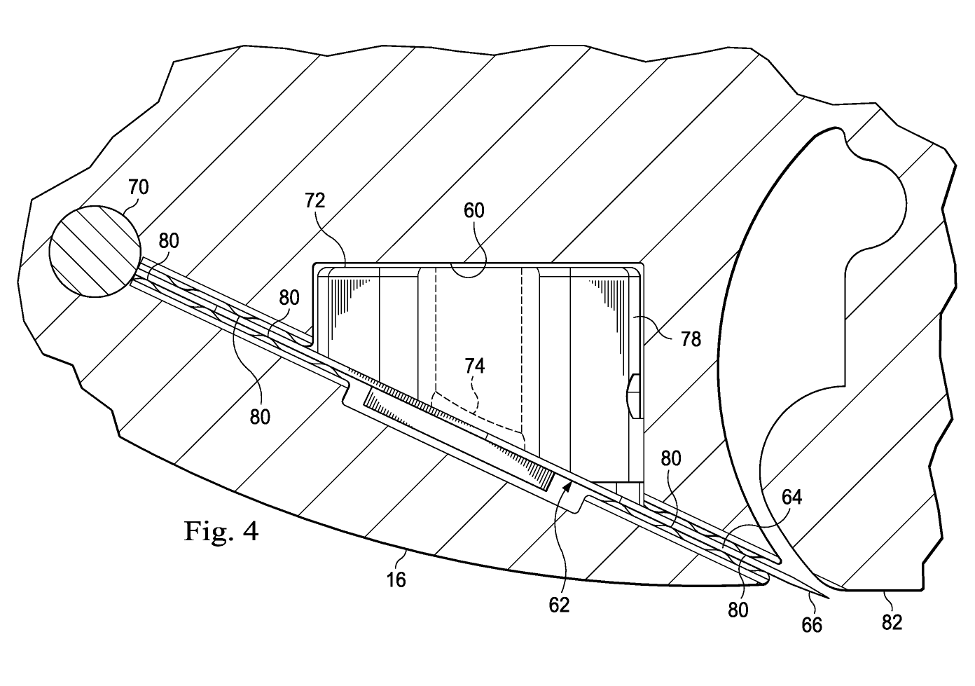

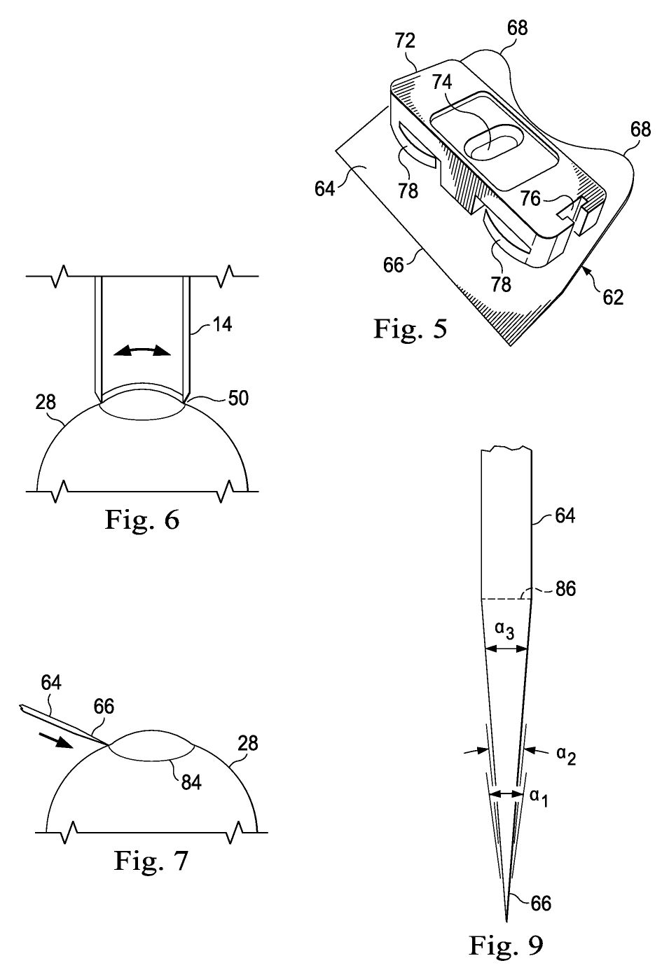

The cutting blade unit 62 can readily be seen in FIG. 5. The unit comprises a planar cutting blade 64, made for example of stainless steel, with a cutting edge 66 on a front blade edge and several (in this case two) bearing sections 68 provided spaced apart from one another on a rear blade edge. Between the bearing sections 68 the rear blade edge is set back. In the illustrated example the bearing sections 68 are rounded; they serve for the support on a convex abutment surface provided in the cutting head 16, which abutment surface can be formed for example by an abutment rod 70 (FIGS. 3, 4). Between the bearing sections 68 of the cutting blade 64 and the abutment rod 70 there exists an approximately punctiform contact, which ensures a low degree of friction and correspondingly low wear.

The cutting blade unit 62 also comprises an attachment 72 on one of the flat blade sides, which for example is made of plastics material and is firmly connected to the cutting blade 64. The attachment 72 is formed with an elongated depression 74 on its upper side remote from the cutting blade 64, in which an eccentric pin (not shown in more detail) of a drive shaft of the aforementioned electric motor drive means engages during operation of the cutting head 16. The cutting blade unit 62 is thereby caused to execute lateral oscillations, which are superimposed on the feed movement of the cutting head 16 effected by the engagement of the drive pinions 38 with the teeth 36.

On at least one of its lateral sides the attachment 72 also comprises an undercut T-shaped groove 76, which serves for the coupling of a slide (not shown in more detail), with which the cutting blade unit 62 is inserted into the receiving pocket 60 of the cutting head 16 and can be removed therefrom after use. The groove 76 thus allows a simple manipulation of the cutting blade unit 62.

The attachment 72 finally also carries spring elements 78, which are arranged on the side of the attachment 72 facing away from the rear blade edge. When the cutting blade unit 62 is inserted into the receiving pocket 60 of the cutting head 16, the spring elements 78 co-operate in such a way with a boundary surface of the receiving pocket 60 as to produce a pretensioning, by means of which the cutting blade unit 62 is pressed against the rear abutment surface of the cutting head 16. In this way a precise positioning and alignment of the cutting blade unit 62 in the receiving pocket 60 is achieved.

As can readily be seen in FIG. 4, the receiving pocket 60, which is open at least on one lateral side of the cutting head 60, is formed having a cross-sectionally enlarged middle section for accommodating the attachment 72 and also two slit-shaped constricted sections lying in front of and behind the latter, in which blade guide formations 80 are provided for the bilateral guidance of the cutting blade 64. The blade guide formations 80 can be formed for example by guide ribs, which extend in the transverse direction of the blade.

The cutting head 16 has an applanation surface 82 arranged in the feed direction in front of the cutting edge 66 of the cutting blade 64, the said applanation surface pressing against the surface of the cornea when the cutting head 16 is in use. The cutting edge 66, which forms a rectilinear cutting edge and thus a further cutting geometry within the meaning of the invention, projects somewhat above the applanation surface 82, in order to be able to penetrate the cornea when the cutting head 16 is being driven.

The parameters influencing the cutting action of the cutting blade 64 are therefore adjusted so that the cutting blade 64 does not damage the Bowman membrane and thus the corneal stroma. This is achieved in particular by a certain bluntness of the cutting edge 66, which is chosen so that although the cutting blade 64 can remove the corneal epithelium from the Bowman membrane, it cannot however penetrate this membrane. Moreover, the setting angle of the cutting blade 64 with respect to the applanation surface 82 and with respect to the feed direction of the cutting head 16 can influence the cutting action. Good results have been achieved with a value of the setting angle of about 25°. Also, the projection of the cutting edge 66 over the applanation surface 82 is conveniently adjusted to be sufficiently small so that it basically corresponds to the desired flap thickness, i.e. roughly to the thickness of the corneal epithelium (typically about 50 to 55 μm).

For reasons of clarity the part of the cutting head 16 lying underneath the cutting blade 64 in FIG. 4 has been omitted in FIG. 3, so that the cutting blade 64 is fully visible when seen from underneath.

FIGS. 6 and 7 show diagrammatically the two essential phases for producing an epithelial flap with the cutting instrument 10. First of all, according to FIG. 6 a circular cut is made with the trepan 14 in the corneal epithelium of the eye 28. For this purpose the trepan 14 is placed on the surface of the cornea and gently rotated clockwise and anticlockwise. The cutting edge 50 of the trepan 14 thereby penetrates the epithelium. The penetration depth of the trepan 14 can be set for example by stop means (not shown in more detail), which act in the axial direction between the trepan 14 and the guide sleeve 44.

The circular cut thus made with the trepan 14 is identified by the reference numeral 84 in FIG. 7. In a following phase of the method for forming the epithelial flap the cutting head 16 is used with the cutting blade 64. During the removal of the trepan 14 and guide sleeve 44 the instrument base body 12 remains held firmly under suction on the eye 28. By driving the cutting head 16 the cutting blade 64 is moved linearly over the eye 28. The cutting blade thereby moves in the previously made circular cut 84 and then lifts off the epithelium from the Bowman membrane, to form the desired flap. It is understood that for this purpose an appropriate matching of the mounting positions of the guide sleeve 44 and trepan 14 on the one hand and of the cutting head 16 on the other hand on the instrument base body 12 is necessary so that the cutting blade 64 engages precisely with the circular cut 84 made by the trepan 14 and can use this as it were as an “entry” to the corneal epithelium.

In the example of implementation described hereinbefore the frame 30 and the guide sleeve 44 form guide holding means in the context of the invention. It is understood that the guide holding means are in no way restricted to such a configuration as is shown in FIGS. 1 and 2, and numerous modifications can be imagined without any problem. The term “guide holding means” should therefore be understood quite generally, so as to include any structures that are capable of holding and movably guiding various cutting units with different cutting geometries on an instrument base body of a micro surgical cutting instrument according to the invention.

Reference will now be made to FIG. 9 with regard to a preferred cutting geometry of the cutting blade 64. This shows in section a portion of the cutting blade 64 in the region of the cutting edge 66. It can be seen that the cutting edge 66 is round. The radius of curvature at the cutting edge 66 is preferably about 5 μm. The cutting edge 66 is located at the end of a blade section, in which the oppositely facing blade sides do not yet run parallel to one another but enclose an angle between them. This angle is here termed the cutting angle. The cutting angle varies in the illustrated example of implementation. In a first cutting region directly adjoining the cutting edge 66 the cutting angle—identified by α₁—is about 18°, in which connection it can deviate preferably only slightly below this value, but most desirably not at all, but can however deviate upwardly by up to 2°. The first cutting region can extend for example over a length of about 0.110 to about 0.15 mm.

In an adjoining second cutting region the cutting angle—here identified by α₂—is smaller than in the first cutting region and is about 13°, in which connection an upward deviation of about 4° is allowable, though preferably no downward deviation is allowable. Finally, a third cutting region is also provided, in which the cutting angle—here identified as α₃—is again smaller than in the previous cutting regions and is about 9°, with a possible upward deviation of about 3°. Downwards the cutting angle α₃ should not be smaller than this value. Overall the blade section from the cutting edge 66 up to the point at which the oppositely facing blade sides become parallel (indicated by the line 86 in FIG. 9) can be about 0.45 to 0.50 mm long.

It is understood that the numerical values mentioned above are given only by way of example and are in no way intended to restrict the invention. However, these numerical values have provided particularly good results.

In FIG. 8 an attachment part 88 can also be seen mounted on the cutting head 16, which serves for the drive-type coupling of the cutting head 16 and of the cutting blade unit 62 inserted therein with electric motor drive means, not shown in more detail.

What is claimed is:

1. Microsurgical cutting instrument for refractive opthalmological treatments, comprising an instrument base body unit to be placed on the eye and which can be fixed relative to the latter, wherein guide holding means are associated with the instrument base body unit, which are intended and designed for the movably guided holding, relative to the instrument base body unit, of at least two cutting units with different cutting geometries.

2. Cutting instrument according to claim 1, characterised in that the guide holding means include at least a first guide formation for guiding a first cutting unit and at least a second guide formation, different from the first guide formation, for guiding a second cutting unit.

3. Cutting instrument according to claim 2, characterised in that the first cutting unit has a circular cutting edge and the at least one first guide formation is designed for the rotational guidance of the first cutting unit, and that the second cutting unit has a rectilinear cutting edge 6 and the at least one second guide formation (36, 46) is designed for the at least approximately linear guidance of the second cutting unit.

4. Cutting instrument according to claim 2, characterised in that the guide holding means permit the simultaneous arrangement of both cutting units on the instrument base body unit.

5. Cutting instrument according to claim 2, characterised in that the guide holding means include a guide body separate from the instrument base body unit but that can be positioned on the latter, that the at least one first guide formation is formed on the guide body, while the at least one second guide formation is formed on the instrument base body unit, and that for the cutting operation of the second guide unit the guide body has to be removed from the instrument base body unit.

6. Cutting instrument according to claim 3, characterised in that the guide body has an approximately cylindrical guide receptacle opening, in which the first cutting unit can be accommodated so as to rotate about the cylindrical axis, and that the guide body and the instrument base body unit have co-operating rotation prevention means, which prevent the guide body rotating about the cylindrical axis relative to the instrument base body unit.

7. Cutting instrument according to claim 3 characterised in that the cutting edge of the second cutting unit is formed by a flat cutting blade, which is arranged at an angle between 18° and 32°, preferably between 21° and 28° and most preferably at an angle of about 25°, to the guiding direction of the second cutting unit.

8. Method for forming a corneal flap on the eye, comprising the following steps:
forming a circular cut in the cornea by means of a first cutting unit comprising a circular cutting edge, and
inserting a rectilinear cutting edge of a second cutting unit into the cornea, starting from the circular cut, in order to produce the flap.

9. Method according to claim 8, in which the sharpness of the rectilinear cutting edge is such that, on inserting the rectilinear cutting edge into the cornea, the Bowman membrane remains substantially undamaged.

10. (canceled)

11. (canceled)

12. The instrument of claim 1, wherein at least one of the cutting units includes a rectilinear cutting edge of circular cross-section, the cutting edge having a radius of curvature of between 1 and 10 μm, and the cutting unit further including a first cutting region adjoining the cutting edge having a cutting angle of between 15° and 22°.

13. The instrument of claim 12, wherein the radius is between 4 and 6 μm and the cutting angle is approximately 18°.

14. The instrument of claim 12, wherein the cutting unit includes a second cutting region adjoining the first cutting region, the second cutting region having a second cutting angle less than the first cutting angle substantially between 10° and 18°.

15. The instrument of claim 14, wherein the second cutting angle is about 13°.

16. The instrument of claim 14, wherein the cutting unit includes a third cutting region adjoining the second cutting region, the third cutting region having a third cutting angle less than the cutting angle of the second cutting region.

17. The method of claim 8, further including providing a first cutting unit having a rectilinear cutting edge of circular cross-section, the cutting edge having a radius of curvature of between 1 and 10 μm, and the cutting unit further including a first cutting region adjoining the cutting edge having a cutting angle of between 15° and 22°.

18. The method of claim 17, wherein said providing further includes providing a cutting unit with a second cutting region having a second cutting angle less than the first cutting region.

19. The method of claim 18, wherein said providing further includes providing a cutting unit with a third cutting region having a third cutting angle less than the second cutting region.
